(19) United States
(12) Patent Application Publication
(10) Pub. No.: US 2014/0316516 A1
(43) Pub. Date: Oct. 23, 2014

(54) MULTI-COMPONENT CUFF DESIGNS FOR TRANSCATHETER MITRAL VALVE REPLACEMENT SUBVALVULAR SEALING APPARATUS FOR TRANSCATHETER MITRAL VALVES AND WIRE FRAMED LEAFLET ASSEMBLY

(71) Applicant: Tendyne Holdings, Inc., Roseville, MN (US)

(72) Inventors: Robert VIDLUND, Forest Lake, MN (US); Kemal SCHANKERELI, Stillwater, MN (US); Michael DAVIS, Shorewood, MN (US)

(73) Assignee: TENDYNE HOLDINGS, INC., Roseville, MN (US)

(21) Appl. No.: 14/322,294

(22) Filed: Jul. 2, 2014

Related U.S. Application Data
(63) Continuation of application No. PCT/US2012/072282, filed on Dec. 31, 2012.
(60) Provisional application No. 61/582,845, filed on Jan. 4, 2012.
(60) Provisional application No. 61/582,842, filed on Jan. 4, 2012.

(51) Int. Cl. A61F 2/24

(52) U.S. Cl. CPC A61F 2/24; A61F 2/2418
USPC 623/2.1

(57) ABSTRACT

This invention relates to improved devices and methods for deploying and sealing a prosthetic cardiac valve, including (i) a mitral valve with a cuff or atrial sealing gasket formed from tissue or fabric-covered wire or laser cut metal originating from one end of an expandable stent, wherein such cuff or gasket is contoured to fill the zone of coaptation between the commissures of the native valve, (ii) an articulating cuff comprised of a series of radially extending tines having a loop terminus to improve sealing a prosthetic mitral valve against hemodynamic leaking, methods of fitting a prosthetic valve with such an articulating cuff wherein such cuff is contoured to fill the zone of coaptation between the commissures of the native valve, and/or (iii) an expandable leaflet assembly comprising tissue or fabric leaflets extending to cover a wire frame of three or more repeating arches.

### CROSS-REFERENCE TO RELATED APPLICATIONS

This application is a continuation of International Application No. PCT/US2012/072282, filed Dec. 31, 2012, which claims priority to and the benefit of U.S. Provisional Application No. 61/582,845, filed Jan. 4, 2012 and U.S. Provisional Application No. 61/582,842, filed Jan. 4, 2012, the disclosures of all of which are incorporated herein by reference in their entirety.

### BACKGROUND

### Field of the Invention

This invention relates to improvements to the cuff design of a transcatheter mitral valve replacement.

Valvular heart disease and specifically aortic and mitral valve disease is a significant health issue in the US Annually approximately 90,000 valve replacements are conducted in the US. Traditional valve replacement surgery, the orthotopic replacement of a heart valve, is an “open heart” surgical procedure. Briefly, the procedure necessitates surgical opening of the thorax, the initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated to the procedure largely due to the necessity for extra-corporeal circulation. Further, open heart surgery is often poorly tolerated in elderly patients.

Thus if the extra-corporeal component of the procedure could be eliminated, morbidities and cost of valve replacement therapies would be significantly reduced.

While replacement of the aortic valve in a transcatheter manner is the subject of intense investigation, lesser attention has been focused on the mitral valve. This is in part reflective of the greater level of complexity associated to the native mitral valve apparatus and thus a greater level of difficulty with regards to inserting and anchoring the replacement prosthesis.

Several designs for catheter-deployed (transcatheter) aortic valve replacement are under various stages of development. The Edwards SAPIEN transcatheter heart valve is currently undergoing clinical trial in patients with calcific aortic valve disease who are considered high-risk for conventional open-heart valve surgery. This valve is deployable via a retrograde transarterial (transfemoral) approach or an antegrade transapical (transventricular) approach. A key aspect of the Edwards SAPIEN and other transcatheter aortic valve replacement designs is their dependence on lateral fixation (e.g. tines) that engages the valve tissues as the primary anchoring mechanism. Such a design basically relies on circumferential friction around the valve housing or stent to prevent dislodgement during the cardiac cycle. This anchoring mechanism is facilitated by, and may somewhat depend on, a calcified aortic valve annulus. This design also requires that the valve housing or stent have a certain degree of rigidity.

At least one transcatheter mitral valve design is currently in development. The Endovalve uses a folding tripod-like design that delivers a tri-leaflet bioprosthetic valve. It is designed to be deployed from a minimally invasive transatrial approach, and could eventually be adapted to a transvenous atrial septotomy delivery. This design uses “proprietary gripping features” designed to engage the valve annulus and leaflets tissues. Thus the anchoring mechanism of this device is essentially equivalent to that used by transcatheter aortic valve replacement designs.

Various problems continue to exist in this field, including problems with insufficient articulation and sealing of the valve within the native annulus, pulmonary edema due to poor atrial drainage, perivalvular leaking around the install prosthetic valve, lack of a good fit for the prosthetic valve within the native mitral annulus, atrial tissue erosion, excess wear on the nitinol structures, interference with the aorta at the posterior side of the mitral annulus, and lack of customization, to name a few. Accordingly, there is still a need for an improved prosthetic mitral valve having a multi-component cuff or atrial sealing gasket, articulating collar support structures and/or a stentless valve comprising tissue or fabric sewn or otherwise formed onto a wire frame, then secured within a stent.

### SUMMARY

The present invention relates to the improved design and function of pre-configured compressible transcatheter prosthetic heart valve having improved multi-component collar which can be deployed into a closed beating heart using a transcatheter delivery system.

In a preferred embodiment, there is provided a pre-configured compressible transcatheter prosthetic heart valve having improved multi-component collar, which comprises an expandable tubular stent and an expandable internal leaflet assembly, said expandable tubular stent having a flared end and a stent body, wherein the improved multi-component collar is comprised of a first collar material and a second collar material, said first and second collar material attached to the flared end of the expandable tubular stent, said first collar material comprised of biocompatible synthetic material, said second collar material comprised of stabilized tissue, wherein said valve having improved multi-component collar locally contours to the mitral structures and/or annulus, and wherein said leaflet assembly is disposed within the stent and is comprised of stabilized tissue or synthetic material.

The design as provided focuses on the deployment of a device via a minimally invasive fashion and by way of example considers a minimally invasive surgical procedure utilizing the intercostal or subxyphoid space for valve introduction, but may also include standard retrograde, or antegrade transcatheter approaches. In order to accomplish this, the valve is formed in such a manner that it can be compressed to fit within a delivery system and secondarily ejected from the delivery system into the target location, for example the mitral or tricuspid valve annulus.

Multi-Component Collar with Stent Variations

In a preferred embodiment, there is provided a prosthetic mitral valve containing an improved stent which locally contours to the mitral structures and/or annulus.

In another preferred embodiment, there is provided a prosthetic heart valve with a stent body that has a low height to width profile.

In a preferred embodiment, the prosthetic mitral valve contains an improved stent body that is a half-round D-shape in cross-section.

In a preferred embodiment, the prosthetic mitral valve contains an improved stent body that is a bent tubular stent structure wherein the bend is directed away from the anterior leaflet, away from interfering with coaptation of adjacent, e.g. aortic, valvular leaflets.

In a preferred embodiment, the prosthetic mitral valve contains an improved stent body that has a low height to width profile and the leaflet structure disposed within the stent is positioned at or near the atrial end of the stent body.

In another preferred embodiment, the a prosthetic mitral valve has a stent body made from both braided wire (atrial end) and laser-cut metal (annular or ventricular end), or vice versa.

### Additional Features for Improved Stents

In a preferred embodiment, the prosthetic heart valve has a cuff that has articulating wire loops of various lengths.

In another preferred embodiment, the prosthetic heart valve has at least one elastic tether to provide compliance during the physiologic movement or conformational changes associated with heart contraction.

In another preferred embodiment, the prosthetic heart valve has a stent body and cuff that are made from a superelastic metal.

In another preferred embodiment, the prosthetic heart valve has a tether which is used to position the valve cuff into the mitral annulus to prevent perivalvular leak.

In another preferred embodiment, the tethers are bioabsorbable and provide temporary anchoring until biological fixation of the prosthesis occurs. Biological fixation consisting of fibrous adhesions between the leaflet tissues and prosthesis or compression on the prosthesis by reversal of heart dilation, or both.

In another preferred embodiment, the prosthetic heart valve has a cuff for a prosthetic heart valve, said cuff being covered with tissue.

In another preferred embodiment, the cuff is covered with a synthetic polymer selected from expandable polytetrafluoroethylene (ePTFE) or polyester.

In another preferred embodiment, there is provided a prosthetic heart valve that has leaflet material constructed from a material selected from the group consisting of polyurethane, polytetrafluoroethylene, pericardium, and small intestine submucosa.

In another preferred embodiment, there is provided a prosthetic heart valve having surfaces that are treated with anticoagulant.

In another preferred embodiment, there is provided a prosthetic heart valve having a cuff and containing anchoring tethers which are attached to the cuff.

In another preferred embodiment, there is provided a prosthetic heart valve having a cuff and containing anchoring tethers which are attached to the cuff and at both commissural tips.

In another preferred embodiment, there is provided a prosthetic heart valve having a cuff where the cuff attachment relative to the body is within the angles of about 60 degrees to about 150 degrees.

In another preferred embodiment, there is provided a prosthetic heart valve containing a combination of tethers and barbs useful for anchoring the device into the mitral annulus.

In another embodiment, the wire of the cuff is formed as a series of radially extending loops of equal or variable length.

In another embodiment, the cuff extends laterally beyond the expanded tubular stent according to a ratio of the relationship between the height of the expanded deployed stent (h) and the lateral distance that the cuff extends onto the tissue (1). Preferably, the h/l ratio can range from 1:10 to 10:1, and more preferably includes without limitation 1:3, 1:2, 1:1, 2:1, and fractional ranges there between such as 1.25:2.0, 1.5:2.0, and so forth. It is contemplated in one non-limiting example that the cuff can extend laterally (1) between about 3 and about 30 millimeters.

In another embodiment, there is provided a feature wherein the tubular stent has a first end and a second end, wherein the cuff is formed from the stent itself, or in the alternative is formed separately and wherein the cuff is located at the first end of the stent, and the second end of the tubular stent has a plurality of tether attachment structures.

In another embodiment, there is provided a feature further comprising a plurality of tethers for anchoring the prosthetic heart valve to tissue and/or for positioning the prosthetic heart valve.

In another embodiment, there is provided a feature further comprising an epicardial tether securing device, wherein the tethers extend from about 2 cm to about 20 cm in length, and are fastened to an epicardial tether securing device. Some pathological conditions within a ventricle may require a atrial-apical tether from about 8 to about 15 cm, or more as described within the range above.

In another embodiment, there is provided a catheter delivery system for delivery of a prosthetic heart valve which comprises a delivery catheter having the prosthetic heart valve disposed therein, and an obturator for expelling the prosthetic heart valve.

In another embodiment, there is provided an assembly kit for preparing the catheter delivery system which comprises a compression funnel, an introducer, a wire snare, an obturator, a delivery catheter, and a prosthetic heart valve, wherein the compression funnel has an aperture for attaching to the introducer, wherein said introducer is comprised of a tube having a diameter that fits within the diameter of the delivery catheter, wherein said obturator is comprised of a tube fitted with a handle at one end and a cap at the other end, wherein said cap has an opening to allow the wire snare to travel therethrough, and said obturator has a diameter that fits within the diameter of the introducer, and wherein said prosthetic heart valve is compressible and fits within the delivery catheter.

In another embodiment, there is provided a method of treating mitral regurgitation and/or tricuspid regurgitation in a patient, which comprises the step of surgically deploying the prosthetic heart valve described herein into the annulus of the target valve structure, e.g. mitral valve annulus and tricuspid valve annulus of the patient.

In another embodiment, there is provided a feature wherein the prosthetic heart valve is deployed by directly accessing the heart through an intercostal space, using an apical approach to enter the left (or right) ventricle, and deploying the prosthetic heart valve into the valvular annulus using the catheter delivery system.

In another embodiment, there is provided a feature wherein the prosthetic heart valve is deployed by directly accessing the heart through a thoracotomy, sternotomy, or minimally-invasive thoracic, thorascopic, or transdiaphragmatic approach to enter the left (or right) ventricle, and deploying the prosthetic heart valve into the valvular annulus using the catheter delivery system.

In another embodiment, there is provided a feature wherein the prosthetic heart valve is deployed by directly accessing the heart through the intercostal space, using a lateral approach to enter the left or right ventricle, and deploying the prosthetic heart valve into the valvular annulus using the catheter delivery system.

In another embodiment, there is provided a feature wherein the prosthetic heart valve is deployed by accessing the left heart using either an antegrade-trans(atrial)septal (transvenous-trans(atrial)septal) approach or a retrograde (transarterial-transaortic) catheter approach to enter the left heart, and deploying the prosthetic heart valve into the mitral annulus using the catheter delivery system.

In another embodiment, there is provided a feature wherein the prosthetic heart valve is deployed into the mitral annulus from a retrograde approach by accessing the left ventricle through the apex of the ventricular septum (transvenous-trans(ventricular)septal approach).

In another embodiment, there is a feature wherein the prosthetic heart valve is deployed into the mitral position using a retrograde transventricular septal approach and the tethers are anchored into or on the right ventricular side of the ventricular septum.

In another embodiment, there is provided a feature further comprising tethering the prosthetic heart valve to tissue within the left ventricle.

In another embodiment, there is provided a feature wherein the prosthetic heart valve is tethered to the apex of the left ventricle using an epicardial tether securing device.

In another embodiment, there is provided a retrieval method for quickly removing a prosthetic heart valve having one or more tethers from a patient using minimally invasive cardiac catheter techniques, which comprises the steps of, capturing the one or more tethers with a catheter having a snare attachment, guiding the captured tethers into a collapsible funnel attachment connected to the removal catheter, pulling the tethers to conform the prosthetic heart valve into a collapsed, compressed conformation, and pulling the now compressed prosthetic heart valve into the removal catheter for subsequent extraction. The retrieval method is contemplated for use for capturing the prosthetic heart valve as described herein or any suitable tethered, collapsible medical device. In a preferred embodiment, the method is used to extract a prosthetic heart valve from either the left or right ventricle. The method may be particularly useful to extract the prosthetic appliance during an aborted surgical deployment.

Multi-Component Collar with Collar Variations

In another preferred embodiment, there is provided a method of sealing a deployed prosthetic mitral valve against hemodynamic leaking, comprising fitting a prosthetic mitral valve with a cuff or atrial sealing gasketprior to deployment wherein the cuff or atrial sealing gasket is constructed to contour to the commissures of a pathologically defective mitral valve and constructed to contour to the zone of coaptation of the pathologically defective mitral valve, wherein the cuff or atrial sealing gasket is formed from wire originating from one end of an expandable tubular braided wire stent and the cuff or atrial sealing gasket is covered with stabilized tissue or synthetic material, the commissural contour components of the cuff or atrial sealing gasket and the zone of coaptation contour components of the cuff or atrial sealing gasket forming a complete or partial saddle-shape wherein the commissural contour components are in direct communication with the mitral valve commissures, and the zone of coaptation contour components are in direct communication with the mitral valve zone of coaptation.

In a preferred embodiment, the cuff or atrial sealing gasket shape is agaricoid.

In another preferred embodiment, the cuff or atrial sealing gasket shape is onychoid.

In another preferred embodiment, the cuff or atrial sealing gasket shape is reniform.

In another preferred embodiment, the cuff or atrial sealing gasket shape is an oval.

In another preferred embodiment, the cuff or atrial sealing gasket shape is a truncated-oval having a squared end.

In another preferred embodiment, the cuff or atrial sealing gasket shape is propeller-shaped having two or three blades.

In another preferred embodiment, the cuff or atrial sealing gasket shape is cruciform.

In another preferred embodiment, the cuff or atrial sealing gasket shape is petal-shaped having flat radial covered loops.

In another preferred embodiment, the cuff or atrial sealing gasket shape is irregular or amoeboid.

In another preferred embodiment, the cuff or atrial sealing gasket shape is cotyloid shaped.

In another preferred embodiment, the cuff or atrial sealing gasket shape is a partial half-round fan-shape.

In another preferred embodiment, the cuff or atrial sealing gasket shape is a rectangular U-shape.

In another preferred embodiment, the cuff or atrial sealing gasket is constructed from ductile metal.

In another preferred embodiment, the cuff or atrial sealing gasket shape is constructed with a cover of stabilized tissue that is derived from adult, or 90-day old, or 30 day old bovine, ovine, equine or porcine pericardium, or from animal small intestine submucosa.

In another preferred embodiment, the cuff or atrial sealing gasket shape is constructed with a cover of synthetic material is selected from the group consisting of polyester, polyurethane, and polytetrafluoroethylene.

In another preferred embodiment, the stabilized tissue or synthetic material is treated with anticoagulant.

In another preferred embodiment, the method further comprises the step of anchoring the prosthetic heart valve to tissue uses a plurality of tethers to the atrial sealing gasket.

In another preferred embodiment, the method further comprises the step of anchoring the prosthetic heart valve to tissue using a single tether attached to the stent or a tether-attachment structure attached to the stent.

In another preferred embodiment, at least one of the plurality of tethers is an elastic tether.

In another preferred embodiment, at least one of the plurality of tethers is a bioresorbable tether.

Another embodiment of the present invention relates to the improved design and function of pre-configured compressible transcatheter prosthetic heart valve having improved articulating collar support structures which can be deployed into a closed beating heart using a transcatheter delivery system.

In a preferred embodiment, there is provided a pre-configured compressible transcatheter prosthetic heart valve having improved articulating collar support structures, which comprises an expandable tubular stent and an expandable internal leaflet assembly, said expandable tubular stent having a flared end and a stent body, wherein the flared end is comprised of a plurality of independent articulating collar support structures, wherein said valve having improved articulating collar support structures locally contours to the mitral structures and/or annulus, and wherein said leaflet assembly is disposed within the stent and is comprised of stabilized tissue or synthetic material.

The design as provided focuses on the deployment of a device via a minimally invasive fashion and by way of example considers a minimally invasive surgical procedure utilizing the intercostal or subxyphoid space for valve introduction, but may also include standard retrograde, or antegrade transcatheter approaches. In order to accomplish this, the valve is formed in such a manner that it can be compressed to fit within a delivery system and secondarily ejected from the delivery system into the target location, for example the mitral or tricuspid valve annulus.

Articulating Collar Support Structures with Stent Variations

In a preferred embodiment, there is provided a prosthetic mitral valve containing an improved stent having a flared collar with independently articulating radial support structures covered with stabilized tissue or synthetic material, or both, which locally contours to the mitral structures and/or annulus.

In another preferred embodiment, there is provided a prosthetic heart valve with a stent body that has a low height to width profile.

In a preferred embodiment, the prosthetic mitral valve contains an improved stent body that is a half-round D-shape in cross-section.

In a preferred embodiment, the prosthetic mitral valve contains an improved stent body that is a bent tubular stent structure wherein the bend is directed away from the anterior leaflet, away from interfering with coaptation of adjacent, e.g. aortic, valvular leaflets.

In a preferred embodiment, the prosthetic mitral valve contains an improved stent body that has a low height to width profile and the leaflet structure disposed within the stent is positioned at or near the atrial end of the stent body.

In another preferred embodiment, the a prosthetic mitral valve has a stent body made from both braided wire (atrial end) and laser-cut metal (annular or ventricular end), or vice versa.

### Additional Features for Improved Stents

In a preferred embodiment, the prosthetic heart valve has a cuff that has articulating wire articulating radial tines or posts of wire of various lengths.

In another preferred embodiment, the prosthetic heart valve has at least one elastic tether to provide compliance during the physiologic movement or conformational changes associated with heart contraction.

In another preferred embodiment, the prosthetic heart valve has a stent body and cuff that are made from a superelastic metal.

In another preferred embodiment, the prosthetic heart valve has a tether which is used to position the valve cuff into the mitral annulus to prevent perivalvular leak.

In another preferred embodiment, the tethers are bioabsorbable and provide temporary anchoring until biological fixation of the prosthesis occurs. Biological fixation consisting of fibrous adhesions between the leaflet tissues and prosthesis or compression on the prosthesis by reversal of heart dilation, or both.

In another preferred embodiment, the prosthetic heart valve has a cuff for a prosthetic heart valve, said cuff being covered with tissue.

In another preferred embodiment, the cuff is covered with a synthetic polymer selected from expandable polytetrafluoroethylene (ePTFE) or polyester.

In another preferred embodiment, there is provided a prosthetic heart valve that has leaflet material constructed from a material selected from the group consisting of polyurethane, polytetrafluoroethylene, pericardium, and small intestine submucosa.

In another preferred embodiment, there is provided a prosthetic heart valve having surfaces that are treated with anticoagulant.

In another preferred embodiment, there is provided a prosthetic heart valve having a cuff and containing anchoring tethers which are attached to the cuff.

In another preferred embodiment, there is provided a prosthetic heart valve having a cuff and containing anchoring tethers which are attached to the cuff and at both commissural tips.

In another preferred embodiment, there is provided a prosthetic heart valve having a cuff where the cuff attachment relative to the body is within the angles of about 60 degrees to about 150 degrees.

In another preferred embodiment, there is provided a prosthetic heart valve containing a combination of tethers and barbs useful for anchoring the device into the mitral annulus.

In another embodiment, the wire of the cuff is formed as a series of radially extending articulating radial tines or posts of wire of equal or variable length.

In another embodiment, the cuff extends laterally beyond the expanded tubular stent according to a ratio of the relationship between the height of the expanded deployed stent (h) and the lateral distance that the cuff extends onto the tissue (1). Preferably, the h/l ratio can range from 1:10 to 10:1, and more preferably includes without limitation 1:3, 1:2, 1:1, 2:1, and fractional ranges there between such as 1.25:2.0, 1.5:2.0, and so forth. It is contemplated in one non-limiting example that the cuff can extend laterally (1) between about 3 and about 30 millimeters.

In another embodiment, there is provided a feature wherein the tubular stent has a first end and a second end, wherein the cuff is formed from the stent itself, or in the alternative is formed separately and wherein the cuff is located at the first end of the stent, and the second end of the tubular stent has a plurality of tether attachment structures.

In another embodiment, there is provided a feature further comprising a plurality of tethers for anchoring the prosthetic heart valve to tissue and/or for positioning the prosthetic heart valve.

In another embodiment, there is provided a feature further comprising an epicardial tether securing device, wherein the tethers extend from about 2 cm to about 20 cm in length, and are fastened to an epicardial tether securing device. Some pathological conditions within a ventricle may require a atrial-apical tether from about 8 to about 15 cm, or more as described within the range above.

In another embodiment, there is provided a catheter delivery system for delivery of a prosthetic heart valve which comprises a delivery catheter having the prosthetic heart valve disposed therein, and an obturator for expelling the prosthetic heart valve.

In another embodiment, there is provided an assembly kit for preparing the catheter delivery system which comprises a compression funnel, an introducer, a wire snare, an obturator, a delivery catheter, and a prosthetic heart valve, wherein the compression funnel has an aperture for attaching to the introducer, wherein said introducer is comprised of a tube having a diameter that fits within the diameter of the delivery catheter, wherein said obturator is comprised of a tube fitted with a handle at one end and a cap at the other end, wherein said cap has an opening to allow the wire snare to travel therethrough, and said obturator has a diameter that fits within the diameter of the introducer, and wherein said prosthetic heart valve is compressible and fits within the delivery catheter.

In another embodiment, there is provided a method of treating mitral regurgitation and/or tricuspid regurgitation in a patient, which comprises the step of surgically deploying the prosthetic heart valve described herein into the annulus of the target valve structure, e.g. mitral valve annulus and tricuspid valve annulus of the patient.

In another embodiment, there is provided a feature wherein the prosthetic heart valve is deployed by directly accessing the heart through an intercostal space, using an apical approach to enter the left (or right) ventricle, and deploying the prosthetic heart valve into the valvular annulus using the catheter delivery system.

In another embodiment, there is provided a feature wherein the prosthetic heart valve is deployed by directly accessing the heart through a thoracotomy, sternotomy, or minimally-invasive thoracic, thorascopic, or transdiaphragmatic approach to enter the left (or right) ventricle, and deploying the prosthetic heart valve into the valvular annulus using the catheter delivery system.

In another embodiment, there is provided a feature wherein the prosthetic heart valve is deployed by directly accessing the heart through the intercostal space, using a lateral approach to enter the left or right ventricle, and deploying the prosthetic heart valve into the valvular annulus using the catheter delivery system.

In another embodiment, there is provided a feature wherein the prosthetic heart valve is deployed by accessing the left heart using either an antegrade-trans(atrial)septal (transvenous-trans(atrial)septal) approach or a retrograde (transarterial-transaortic) catheter approach to enter the left heart, and deploying the prosthetic heart valve into the mitral annulus using the catheter delivery system.

In another embodiment, there is provided a feature wherein the prosthetic heart valve is deployed into the mitral annulus from a retrograde approach by accessing the left ventricle through the apex of the ventricular septum (transvenous-trans(ventricular)septal approach).

In another embodiment, there is a feature wherein the prosthetic heart valve is deployed into the mitral position using a retrograde transventricular septal approach and the tethers are anchored into or on the right ventricular side of the ventricular septum.

In another embodiment, there is provided a feature further comprising tethering the prosthetic heart valve to tissue within the left ventricle.

In another embodiment, there is provided a feature wherein the prosthetic heart valve is tethered to the apex of the left ventricle using an epicardial tether securing device.

In another embodiment, there is provided a retrieval method for quickly removing a prosthetic heart valve having one or more tethers from a patient using minimally invasive cardiac catheter techniques, which comprises the steps of, capturing the one or more tethers with a catheter having a snare attachment, guiding the captured tethers into a collapsible funnel attachment connected to the removal catheter, pulling the tethers to conform the prosthetic heart valve into a collapsed, compressed conformation, and pulling the now compressed prosthetic heart valve into the removal catheter for subsequent extraction. The retrieval method is contemplated for use for capturing the prosthetic heart valve as described herein or any suitable tethered, collapsible medical device. In a preferred embodiment, the method is used to extract a prosthetic heart valve from either the left or right ventricle. The method may be particularly useful to extract the prosthetic appliance during an aborted surgical deployment.

Articulating Collar Support Structures with Collar Variations

In another preferred embodiment, there is provided a method of sealing a deployed prosthetic mitral valve against hemodynamic leaking, comprising fitting a prosthetic mitral valve with a cuff or atrial sealing gasket having articulating collar support structures prior to deployment wherein the cuff or atrial sealing gasket is constructed to contour to the commissures of a pathologically defective mitral valve and constructed to contour to the zone of coaptation of the pathologically defective mitral valve, wherein the cuff or atrial sealing gasket is formed from wire originating from one end of an expandable tubular braided wire stent and the cuff or atrial sealing gasket is covered with stabilized tissue or synthetic material, the commissural contour components of the cuff or atrial sealing gasket and the zone of coaptation contour components of the cuff or atrial sealing gasket forming a complete or partial saddle-shape wherein the commissural contour components are in direct communication with the mitral valve commissures, and the zone of coaptation contour components are in direct communication with the mitral valve zone of coaptation.

In a preferred embodiment, the cuff or atrial sealing gasket shape is agaricoid.

In another preferred embodiment, the cuff or atrial sealing gasket shape is onychoid.

In another preferred embodiment, the cuff or atrial sealing gasket shape is reniform.

In another preferred embodiment, the cuff or atrial sealing gasket shape is an oval.

In another preferred embodiment, the cuff or atrial sealing gasket shape is a truncated-oval having a squared end.

In another preferred embodiment, the cuff or atrial sealing gasket shape is propeller-shaped having two or three blades.

In another preferred embodiment, the cuff or atrial sealing gasket shape is cruciform.

In another preferred embodiment, the cuff or atrial sealing gasket shape is petal-shaped having flat radial covered articulating radial tines or posts of wire.

In another preferred embodiment, the cuff or atrial sealing gasket shape is irregular or amoeboid.

In another preferred embodiment, the cuff or atrial sealing gasket shape is cotyloid shaped.

In another preferred embodiment, the cuff or atrial sealing gasket shape is a partial half-round fan-shape.

In another preferred embodiment, the cuff or atrial sealing gasket shape is a rectangular U-shape.

In another preferred embodiment, the cuff or atrial sealing gasket is constructed from ductile metal.

In another preferred embodiment, the cuff or atrial sealing gasket shape is constructed with a cover of stabilized tissue that is derived from adult, or 90-day old, or 30 day old bovine, ovine, equine or porcine pericardium, or from animal small intestine submucosa.

In another preferred embodiment, the cuff or atrial sealing gasket shape is constructed with a cover of synthetic material is selected from the group consisting of polyester, polyurethane, and polytetrafluoroethylene.

In another preferred embodiment, the stabilized tissue or synthetic material is treated with anticoagulant.

In another preferred embodiment, the method further comprises the step of anchoring the prosthetic heart valve to tissue uses a plurality of tethers to the atrial sealing gasket.

In another preferred embodiment, the method further comprises the step of anchoring the prosthetic heart valve to tissue using a single tether attached to the stent or a tether-attachment structure attached to the stent.

In another preferred embodiment, at least one of the plurality of tethers is an elastic tether.

In another preferred embodiment, at least one of the plurality of tethers is a bioresorbable tether.

### Wire-Framed Leaflet Assembly

In another embodiment, a pre-configured compressible leaflet assembly comprised of stabilized tissue sewn or otherwise covering an expandable wire frame, wherein said frame is comprised of three or more interlocking arches

The leaflet assembly as described, further comprising wherein each side of each arch in the wire frame is formed as a shallow “S” shape.

The leaflet assembly as described, further comprising wherein the interlocking arches of the wire frame are connected via spring-form circular wire components.

The leaflet assembly as described, further comprising wherein the spring-form circular wire component located at the upper point of connection between each arch is comprised of three upwardly stacked circles of equal diameter.

The leaflet assembly as described, further comprising wherein a spring-form circular wire component comprised of a single circle is located at the base of each arch.

The leaflet assembly as described, further comprising wherein the wire frame is made from superelastic metal.

The leaflet assembly as described, further comprising wherein the superelastic metal is a nickel-titanium alloy.

The leaflet assembly as described, wherein the assembly comprises either two or three leaflets.

The leaflet assembly as described, wherein the stabilized tissue is derived from adult, 90-day old, or 30 day old, bovine, ovine, equine or porcine pericardium, or from animal small intestine submucosa.

The leaflet assembly as described, comprising a synthetic material selected from the group consisting of polyester, polyurethane, and polytetrafluoroethylene, in lieu of stabilized tissue.

In another preferred embodiment, a prosthetic heart valve of comprising the leaflet assembly as described, further comprising wherein such leaflet assembly is secured within an an expandable tubular stent.

In another preferred embodiment, the expandable leaflet assembly as described, further comprising wherein such leaflet assembly is a component of any of the prosthetic heart valve designs described herein.

### BRIEF DESCRIPTION OF THE DRAWINGS

Referring now to the FIGURES, FIG. 1 is a top view of one embodiment showing the multi-component cuff or atrial sealing gasket wherein the inner section is synthetic material such as PET or similar fabric, and the outer section is composed of stabilized tissue. FIG. 1 shows the valve without the leaflets installed. FIG. 1 shows how the collar is composed of at least two sections, and inner and an outer section. Here, the inner section is made from synthetic material, such as PET or Dacron®. The outer section is made from stabilized tissue.

FIG. 2 is a side view of one embodiment showing the atrial sealing gasket wherein the the inner connecting section is synthetic material such as PET or similar fabric, and the outer cuff section is composed of stabilized tissue. FIG. 2 shows an embodiment wherein the stent body may not have any covering material. However, it is contemplated that the stent body may be covered with stabilized tissue, synthetic material, or both. When both are used, the layers may be attached to improve in-growth, and/or improve hemodynamic effects.

FIG. 3 is a perspective view of one embodiment showing the multi-component cuff or atrial sealing gasket wherein the inner section is synthetic material such as PET or similar fabric, and the outer section is composed of stabilized tissue. FIG. 3 shows the valve without the leaflets installed. FIG. 1 shows how the collar is composed of at least two sections, and inner and an outer section. Here, the inner section is made from synthetic material, such as PET or Dacron®. The outer section is made from stabilized tissue. The inner fabric/synthetic material section is important in preferred embodiments in order to facilitate ingrowth once the valve is deployed within the patient.

FIG. 4 is a side view of one embodiment showing the articulating collar support structures of the flared end of the tubular stent. Note this figure does not illustrate the final valve product as it has neither the surface coatings, e.g. synthetic material and/or stabilized tissue, nor internal leaflet structures have been added.

FIG. 5 is a top view of one embodiment showing the articulating collar support structures of the flared end of the tubular stent. Note this figure does not illustrate the final valve product as it has neither the surface coatings, e.g. synthetic material and/or stabilized tissue, nor internal leaflet structures have been added.

FIG. 6 is a perspective view of one embodiment showing the articulating collar support structures of the flared end of the tubular stent. Note this figure does not illustrate the final valve product as it has neither the surface coatings, e.g. synthetic material and/or stabilized tissue, nor internal leaflet structures have been added.

FIG. 7 is an illustration of a close-up of the stent construction at the junction where the stent body transitions to the articulating radial structures. FIG. 4 shows detail of a compacted, or un-expanded (not deployed) stent structure.

FIG. 8 is an illustration of a side view with false-transparent detail of the inner leaflet structure location of an embodiment of the present invention showing how the articulating feature may be used to create a valve having the leaflet structures high in the stent body and raised into the atrial space while maintaining a collar support and sealing originating lower on the stent body.

FIG. 9 is an illustration of a perspective view of a prosthetic leaflet assembly comprising stabilized tissue or fabric over an expandable wire frame.

FIG. 10 is an illustration of an expandable prosthetic leaflet assembly comprising tissue or fabric covering applied over a wire frame, wherein the leaflet assembly is centered within a metallic stent.

### DETAILED DESCRIPTION

### Functions of the Atrial Sealing Gasket

The cuff or atrial sealing gasket functions in a variety of ways. The first function of the cuff or atrial sealing gasket is to inhibit perivalvular leak/regurgitation of blood around the prosthesis. By flexing and sealing across the irregular contours of the annulus and atrium, leaking is minimized and/or prevented.

The second function of the cuff or atrial sealing gasket is to provide an adjustable and/or compliant bioprosthetic valve. The heart and its structures undergo complex conformational changes during the cardiac cycle. For example, the mitral valve annulus has a complex geometric shape known as a hyperbolic parabloid much like a saddle, with the horn being anterior, the seat back being posterior, and the left and right valleys located medially and laterally. Beyond this complexity, the area of the mitral annulus changes over the course of the cardiac cycle. Further, the geometry of the tricuspid valve and tricuspid annulus continues to be a topic of research, posing its own particular problems. Accordingly, compliance is a very important but unfortunately often overlooked requirement of cardiac devices. Compliance here refers to the ability of the valve to maintain structural position and integrity during the cardiac cycle. Compliance with the motion of the heart is a particularly important feature, especially the ability to provide localized compliance where the underlying surfaces are acting differently from the adjacent surfaces. This ability to vary throughout the cardiac cycle allows the valve to remain seated and properly deployed in a manner not heretofore provided.

Additionally, compliance may be achieved through the use of the tethers where the tethers are preferably made from an elastic material. Tether-based compliance may be used alone, or in combination with the atrial sealing gasket-based compliance.

The third function of the cuff or atrial sealing gasket and valve is to provide a valve that, during surgery, is able to be seated and be able to contour to the irregular surfaces of the atrium. The use of independent tethers allows for side to side fitting of the valve within the annulus. For example, where three tethers are used, they are located circumferentially about 120 degrees relative to each other which allows the surgeon to observe whether or where perivalvular leaking might be occurring and to pull on one side or the other to create localized pressure and reduce or eliminate the leaking.

The fourth function of the cuff or atrial sealing gasket is to counter the forces that act to displace the prosthesis toward/into the ventricle (i.e. atrial pressure and flow-generated shear stress) during ventricular filling.

Additional features of the cuff or atrial sealing gasket include that it functions to strengthen the leaflet assembly/stent combination by providing additional structure. Further, during deployment, the cuff or atrial sealing gasket functions to guide the entire structure, the prosthetic valve, into place at the mitral annulus during deployment and to keep the valve in place once it is deployed. Another important function is to reduce pulmonary edema by improving atrial drainage.

### Cuff or Atrial Sealing Gasket Structure

The cuff or atrial sealing gasket is a substantially flat plate that projects beyond the diameter of the tubular stent to form a rim or border. As used herein, the term atrial sealing gasket, cuff, flange, collar, bonnet, apron, or skirting are considered to be functionally equivalent. When the tubular stent is pulled through the mitral valve aperture, the mitral annulus, by the tether loops in the direction of the left ventricle, the cuff or atrial sealing gasket acts as a collar to stop the tubular stent from traveling any further through the mitral valve aperture. The entire prosthetic valve is held by longitudinal forces between the cuff or atrial sealing gasket which is seated in the left atrium and mitral annulus, and the ventricular tethers attached to the left ventricle.

The cuff or atrial sealing gasket is formed from a stiff, flexible shape-memory material such as the nickel-titanium alloy material Nitinol™ wire that is covered by stabilized tissue or other suitable biocompatible or synthetic material. In one embodiment, the cuff or atrial sealing gasket wire form is constructed from independent articulating radial tines or posts of wire extending axially around the circumference of the bend or seam where the cuff or atrial sealing gasket transitions to the tubular stent (in an integral atrial sealing gasket) or where the cuff or atrial sealing gasket is attached to the stent (where they are separate, but joined components).

Once covered by stabilized tissue or material, the articulating radial tines or posts of wire provide the cuff or atrial sealing gasket the ability to travel up and down, to articulate, along the longitudinal axis that runs through the center of the tubular stent. In other words, the individual articulating radial tines or posts of wire can independently move up and down, and can spring back to their original position due to the relative stiffness of the wire. The tissue or material that covers the cuff or atrial sealing gasket wire has a certain modulus of elasticity such that, when attached to the wire of the atrial sealing gasket, is able to allow the wire spindles to move. This flexibility gives the atrial sealing gasket, upon being deployed within a patient's heart, the ability to conform to the anatomical shape necessary for a particular application. In the example of a prosthetic mitral valve, the cuff or atrial sealing gasket is able to conform to the irregularities of the left atrium and shape of the mitral annulus, and to provide a tight seal against the atrial tissue adjacent the mitral annulus and the tissue within the mitral annulus. As stated previously, this feature importantly provides a degree of flexibility in sizing the a mitral valve and prevents blood from leaking around the implanted prosthetic heart valve.

An additional important aspect of the cuff or atrial sealing gasket dimension and shape is that, when fully seated and secured, the edge of the cuff or atrial sealing gasket preferably should not be oriented laterally into the atrial wall such that it can produce a penetrating or cutting action on the atrial wall.

In one preferred embodiment, the wire spindles of the cuff or atrial sealing gasket are substantially uniform in shape and size. In another preferred embodiment of the present invention, each loop or spindle may be of varying shapes and sizes. In this example, it is contemplated that the articulating radial tines or posts of wire may form a pattern of alternating large and small articulating radial tines or posts of wire, depending on where the valve is being deployed. In the case of a prosthetic mitral valve, pre-operative imaging may allow for customizing the structure of the cuff or atrial sealing gasket depending on a particular patient's anatomical geometry in the vicinity of the mitral annulus.

The cuff or atrial sealing gasket wire form is constructed so as to provide sufficient structural integrity to withstand the intracardiac forces without collapsing. The cuff or atrial sealing gasket wire form is preferably constructed of a superelastic metal, such as Nitinol™® and is capable of maintaining its function as a sealing collar for the tubular stent while under longitudinal forces that might cause a structural deformation or valve displacement. It is contemplated as within the scope of the invention to optionally use other shape memory alloys such as Cu—Zn—Al—Ni alloys, and Cu—Al—Ni alloys. The heart is known to generate an average left atrial pressure between about 8 and 30 mm Hg (about 0.15 to 0.6 psi). This left atrial filling pressure is the expected approximate pressure that would be exerted in the direction of the left ventricle when the prosthesis is open against the outer face of the cuff or atrial sealing gasket as an anchoring force holding the cuff or atrial sealing gasket against the atrial tissue that is adjacent the mitral valve. The cuff or atrial sealing gasket counteracts this longitudinal pressure against the prosthesis in the direction of the left ventricle to keep the valve from being displaced or slipping into the ventricle. In contrast, left ventricular systolic pressure, normally about 120 mm Hg, exerts a force on the closed prosthesis in the direction of the left atrium. The tethers counteract this force and are used to maintain the valve position and withstand the ventricular force during ventricular contraction or systole.

Accordingly, the cuff or atrial sealing gasket has sufficient structural integrity to provide the necessary tension against the tethers without being dislodged and pulled into the left ventricle. After a period of time, changes in the geometry of the heart and/or fibrous adhesion between prosthesis and surrounding cardiac tissues may assist or replace the function of the ventricular tethers in resisting longitudinal forces on the valve prosthesis during ventricular contraction.

### Stent Structure

Preferably, superelastic metal wire, such as Nitinol™ wire, is used for the stent, for the inner wire-based leaflet assembly that is disposed within the stent, and for the cuff or atrial sealing gasket wire form. As stated, it is contemplated as within the scope of the invention to optionally use other shape memory alloys such as Cu—Zn—Al—Ni alloys, and Cu—Al—Ni alloys. It is contemplated that the stent may be constructed as a braided stent or as a laser cut stent. Such stents are available from any number of commercial manufacturers, such as Pulse Systems. Laser cut stents are preferably made from Nickel-Titanium (Nitinol™), but also without limitation made from stainless steel, cobalt chromium, titanium, and other functionally equivalent metals and alloys, or Pulse Systems braided stent that is shape-set by heat treating on a fixture or mandrel.

One key aspect of the stent design is that it be compressible and when released have the stated property that it return to its original (uncompressed) shape. This requirement limits the potential material selections to metals and plastics that have shape memory properties. With regards to metals, Nitinol has been found to be especially useful since it can be processed to be austhenitic, martensitic or super elastic. Martensitic and super elastic alloys can be processed to demonstrate the required compression features.

### Laser Cut Stent

One possible construction of the stent envisions the laser cutting of a thin, isodiametric Nitinol tube. The laser cuts form regular cutouts in the thin Nitinol tube.

Secondarily the tube is placed on a mold of the desired shape, heated to the Martensitic temperature and quenched. The treatment of the stent in this manner will form a stent or stent/cuff or atrial sealing gasket that has shape memory properties and will readily revert to the memory shape at the calibrated temperature.

### Braided Wire Stent

A stent can be constructed utilizing simple braiding techniques. Using a Nitinol wire—for example a 0.012″ wire—and a simple braiding fixture, the wire is wound on the braiding fixture in a simple over/under braiding pattern until an isodiametric tube is formed from a single wire. The two loose ends of the wire are coupled using a stainless steel or Nitinol coupling tube into which the loose ends are placed and crimped. Angular braids of approximately 60 degrees have been found to be particularly useful. Secondarily, the braided stent is placed on a shaping fixture and placed in a muffle furnace at a specified temperature to set the stent to the desired shape and to develop the martensitic or super elastic properties desired.

The stent as envisioned in one preferred embodiment is designed such that the ventricular aspect of the stent comes to 2-5 points onto which anchoring sutures are affixed. The anchoring sutures (tethers) will traverse the ventricle and ultimately be anchored to the epicardial surface of the heart approximately at the level of the apex. The tethers when installed under slight tension will serve to hold the valve in place, i e inhibit paravalvular leakage during systole.

### Leaflet and Assembly Structure

The valve leaflets are held by, or within, a leaflet assembly. In one preferred embodiment of the invention, the leaflet assembly comprises a leaflet wire support structure to which the leaflets are attached and the entire leaflet assembly is housed within the stent body. In this embodiment, the assembly is constructed of wire and stabilized tissue to form a suitable platform for attaching the leaflets. In this aspect, the wire and stabilized tissue allow for the leaflet structure to be compressed when the prosthetic valve is compressed within the deployment catheter, and to spring open into the proper functional shape when the prosthetic valve is opened during deployment. In this embodiment, the leaflet assembly may optionally be attached to and housed within a separate cylindrical liner made of stabilized tissue or material, and the liner is then attached to line the interior of the stent body.

In this embodiment, the leaflet wire support structure is constructed to have a collapsible/expandable geometry. In a preferred embodiment, the structure is a single piece of wire. The wireform is, in one embodiment, constructed from a shape memory alloy such as Nitinol. The structure may optionally be made of a plurality of wires, including between 2 to 10 wires. Further, the geometry of the wire form is without limitation, and may optionally be a series of parabolic inverted collapsible arches to mimic the saddle-like shape of the native annulus when the leaflets are attached. Alternatively, it may optionally be constructed as collapsible concentric rings, or other similar geometric forms that are able to collapse/compress which is followed by an expansion to its functional shape. In certain preferred embodiments, there may be 2, 3 or 4 arches. In another embodiment, closed circular or ellipsoid structure designs are contemplated. In another embodiment, the wire form may be an umbrella-type structure, or other similar unfold-and-lock-open designs. A preferred embodiment utilizes super elastic Nitinol wire approximately 0.015″ in diameter. In this embodiment, the wire is wound around a shaping fixture in such a manner that 2-3 commissural posts are formed. The fixture containing the wrapped wire is placed in a muffle furnace at a pre-determined temperature to set the shape of the wire form and to impart it's super elastic properties. Secondarily, the loose ends of the wireform are joined with a stainless steel or Nitinol tube and crimped to form a continuous shape. In another preferred embodiment, the commissural posts of the wireform are adjoined at their tips by a circular connecting ring, or halo, whose purpose is to minimize inward deflection of the post(s).

In another preferred embodiment, the leaflet assembly is constructed solely of stabilized tissue or other suitable material without a separate wire support structure. The leaflet assembly in this embodiment is also disposed within the lumen of the stent and is attached to the stent to provide a sealed joint between the leaflet assembly and the inner wall of the stent. By definition, it is contemplated within the scope of the invention that any structure made from stabilized tissue and/or wire(s) related to supporting the leaflets within the stent constitute a leaflet assembly. In this embodiment, stabilized tissue or suitable material may also optionally be used as a liner for the inner wall of the stent and is considered part of the leaflet assembly.

Liner tissue or biocompatible material may be processed to have the same or different mechanical qualities, e.g. thickness, durability, etc. from the leaflet tissue.

### Deployment Within the Valvular Annulus

The prosthetic heart valve is, in one embodiment, apically delivered through the apex of the left ventricle of the heart using a catheter system. In one aspect of the apical delivery, the catheter system accesses the heart and pericardial space by intercostal delivery. In another delivery approach, the catheter system delivers the prosthetic heart valve using either an antegrade or retrograde delivery approach using a flexible catheter system, and without requiring the rigid tube system commonly used. In another embodiment, the catheter system accesses the heart via a trans-septal approach.

In one non-limiting preferred embodiment, the stent body extends into the ventricle about to the edge of the open mitral valve leaflets (approximately 25% of the distance between the annulus and the ventricular apex). The open native leaflets lay against the outside stent wall and parallel to the long axis of the stent (i.e. the stent holds the native mitral valve open).

In one non-limiting preferred embodiment, the diameter should approximately match the diameter of the mitral annulus. Optionally, the valve may be positioned to sit in the mitral annulus at a slight angle directed away from the aortic valve such that it is not obstructing flow through the aortic valve. Optionally, the outflow portion (bottom) of the stent should not be too close to the lateral wall of the ventricle or papillary muscle as this position may interfere with flow through the prosthesis. As these options relate to the tricuspid, the position of the tricuspid valve may be very similar to that of the mitral valve.

In another embodiment, the prosthetic valve is sized and configured for use in areas other than the mitral annulus, including, without limitation, the tricuspid valve between the right atrium and right ventricle. Alternative embodiments may optionally include variations to the cuff or atrial sealing gasket structure to accommodate deployment to the pulmonary valve between the right ventricle and pulmonary artery, and the aortic valve between the left ventricle and the aorta. In one embodiment, the prosthetic valve is optionally used as a venous backflow valve for the venous system, including without limitation the vena cava, femoral, subclavian, pulmonary, hepatic, renal and cardiac. In this aspect, the cuff or atrial sealing gasket feature is utilized to provide additional protection against leaking

### Tethers

In one preferred embodiment, there are tethers attached to the prosthetic heart valve that extend to one or more tissue anchor locations within the heart. In one preferred embodiment, the tethers extend downward through the left ventricle, exiting the left ventricle at the apex of the heart to be fastened on the epicardial surface outside of the heart. Similar anchoring is contemplated herein as it regards the tricuspid, or other valve structure requiring a prosthetic. There may be from 1 to 8 tethers which are preferably attached to the stent.

In another preferred embodiment, the tethers may optionally be attached to the cuff or atrial sealing gasket to provide additional control over position, adjustment, and compliance. In this preferred embodiment, one or more tethers are optionally attached to the atrial sealing gasket, in addition to, or optionally, in place of, the tethers attached to the stent. By attaching to the cuff or atrial sealing gasket and/or the stent, an even higher degree of control over positioning, adjustment, and compliance is provided to the operator during deployment.

During deployment, the operator is able to adjust or customize the tethers to the correct length for a particular patient's anatomy. The tethers also allow the operator to tighten the cuff or atrial sealing gasket onto the tissue around the valvular annulus by pulling the tethers, which creates a leak-free seal.

In another preferred embodiment, the tethers are optionally anchored to other tissue locations depending on the particular application of the prosthetic heart valve. In the case of a mitral valve, or the tricuspid valve, there are optionally one or more tethers anchored to one or both papillary muscles, septum, and/or ventricular wall.

The tethers, in conjunction with the atrial sealing gasket, provide for a compliant valve which has heretofore not been available. The tethers are made from surgical-grade materials such as biocompatible polymer suture material. Non-limiting examples of such material include ultra high-molecular weight polyethylene (UHMWPE), 2-0 exPFTE (polytetrafluoroethylene) or 2-0 polypropylene. In one embodiment the tethers are inelastic. It is also contemplated that one or more of the tethers may optionally be elastic to provide an even further degree of compliance of the valve during the cardiac cycle. Upon being drawn to and through the apex of the heart, the tethers may be fastened by a suitable mechanism such as tying off to a pledget or similar adjustable button-type anchoring device to inhibit retraction of the tether back into the ventricle. It is also contemplated that the tethers might be bioresorbable/bioabsorbable and thereby provide temporary fixation until other types of fixation take hold such a biological fibrous adhesion between the tissues and prosthesis and/or radial compression from a reduction in the degree of heart chamber dilation.

Further, it is contemplated that the prosthetic heart valve may optionally be deployed with a combination of installation tethers and permanent tethers, attached to either the stent or atrial sealing gasket, or both, the installation tethers being removed after the valve is successfully deployed. It is also contemplated that combinations of inelastic and elastic tethers may optionally be used for deployment and to provide structural and positional compliance of the valve during the cardiac cycle.

### Pledget

In one embodiment, to control the potential tearing of tissue at the apical entry point of the delivery system, a circular, semi-circular, or multi-part pledget is employed. The pledget may be constructed from a semi-rigid material such as PFTE felt. Prior to puncturing of the apex by the delivery system, the felt is firmly attached to the heart such that the apex is centrally located. Secondarily, the delivery system is introduced through the central area, or orifice as it may be, of the pledget. Positioned and attached in this manner, the pledget acts to control any potential tearing at the apex.

### Tines/Barbs

In another embodiment the valve can be seated within the valvular annulus through the use of tines or barbs. These may be used in conjunction with, or in place of one or more tethers. The tines or barbs are located to provide attachment to adjacent tissue. In one preferred embodiment, the tines are optionally circumferentially located around the bend/transition area between the stent and the atrial sealing gasket. Such tines are forced into the annular tissue by mechanical means such as using a balloon catheter. In one non-limiting embodiment, the tines may optionally be semi-circular hooks that upon expansion of the stent body, pierce, rotate into, and hold annular tissue securely.

### Stabilized Tissue or Biocompatible Material

In one embodiment, it is contemplated that multiple types of tissue and biocompatible material may be used to cover the atrial sealing gasket,to form the valve leaflets, to form a wireless leaflet assembly, and/or to line both the inner and/or outer lateral walls of the stent. As stated previously, the leaflet component may be constructed solely from stabilized tissue, without using wire, to create a leaflet assembly and valve leaflets. In this aspect, the tissue-only leaflet component may be attached to the stent with or without the use of the wire form. In a preferred embodiment, there can be anywhere from 1, 2, 3 or 4 leaflets, or valve cusps.

It is contemplated that the tissue may be used to cover the inside of the stent body, the outside of the stent body, and the top and/or bottom side of the cuff or atrial sealing gasket wire form, or any combination thereof.

In one preferred embodiment, the tissue used herein is optionally a biological tissue and may be a chemically stabilized valve of an animal, such as a pig. In another preferred embodiment, the biological tissue is used to make leaflets that are sewn or attached to a metal frame. This tissue is chemically stabilized pericardial tissue of an animal, such as a cow (bovine pericardium) or sheep (ovine pericardium) or pig (porcine pericardium) or horse (equine pericardium).

Preferably, the tissue is bovine pericardial tissue. Examples of suitable tissue include that used in the products Duraguard®, Peri-Guard®, and Vascu-Guard®, all products currently used in surgical procedures, and which are marketed as being harvested generally from cattle less than 30 months old. Other patents and publications disclose the surgical use of harvested, biocompatible animal thin tissues suitable herein as biocompatible “jackets” or sleeves for implantable stents, including for example, U.S. Pat. No. 5,554,185 to Block, U.S. Pat. No. 7,108,717 to Design & Performance-Cyprus Limited disclosing a covered stent assembly, U.S. Pat. No. 6,440,164 to Scimed Life Systems, Inc. disclosing a bioprosthetic valve for implantation, and U.S. Pat. No. 5,336,616 to LifeCell Corporation discloses acellular collagen-based tissue matrix for transplantation.

In one preferred embodiment, the valve leaflets may optionally be made from a synthetic material such a polyurethane or polytetrafluoroethylene. Where a thin, durable synthetic material is contemplated, e.g. for covering the atrial sealing gasket, synthetic polymer materials such expanded polytetrafluoroethylene or polyester may optionally be used. Other suitable materials may optionally include thermoplastic polycarbonate urethane, polyether urethane, segmented polyether urethane, silicone polyether urethane, silicone-polycarbonate urethane, and ultra-high molecular weight polyethylene. Additional biocompatible polymers may optionally include polyolefins, elastomers, polyethylene-glycols, polyethersulphones, polysulphones, polyvinylpyrrolidones, polyvinylchlorides, other fluoropolymers, silicone polyesters, siloxane polymers and/or oligomers, and/or polylactones, and block co-polymers using the same.

In another embodiment, the valve leaflets may optionally have a surface that has been treated with (or reacted with) an anti-coagulant, such as, without limitation, immobilized heparin. Such currently available heparinized polymers are known and available to a person of ordinary skill in the art.

Alternatively, the valve leaflets may optionally be made from pericardial tissue or small intestine submucosal tissue.

### DESCRIPTION OF FIGURES

Referring now to the FIGURES, FIG. 1 is a top view of one embodiment showing the multi-component cuff or atrial sealing gasket wherein the inner section is synthetic material such as PET or similar fabric, and the outer section is composed of stabilized tissue. FIG. 1 shows the valve without the leaflets installed. FIG. 1 shows how the collar is composed of at least two sections, and inner and an outer section. Here, the inner section is made from synthetic material, such as PET or Dacron®. The outer section is made from stabilized tissue.

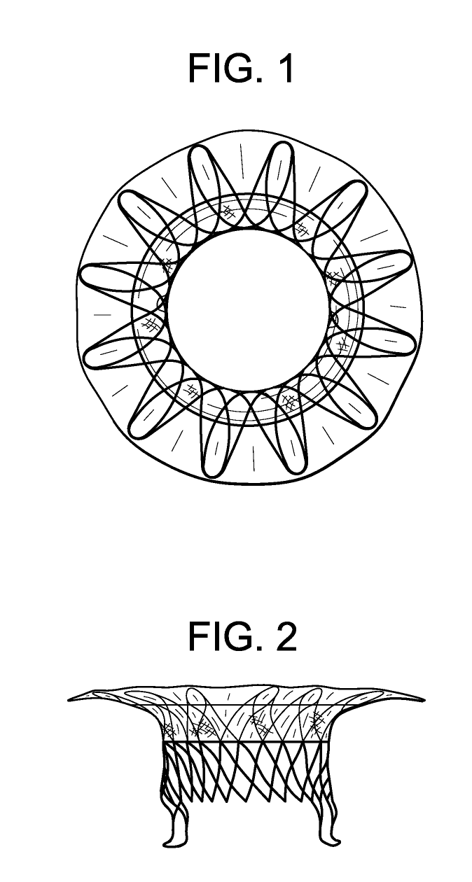

FIG. 2 is a side view of one embodiment showing the atrial sealing gasket wherein the the inner connecting section is synthetic material such as PET or similar fabric, and the outer cuff section is composed of stabilized tissue. FIG. 2 shows an embodiment wherein the stent body may not have any covering material. However, it is contemplated that the stent body may be covered with stabilized tissue, synthetic material, or both. When both are used, the layers may be attached to improve in-growth, and/or improve hemodynamic effects.

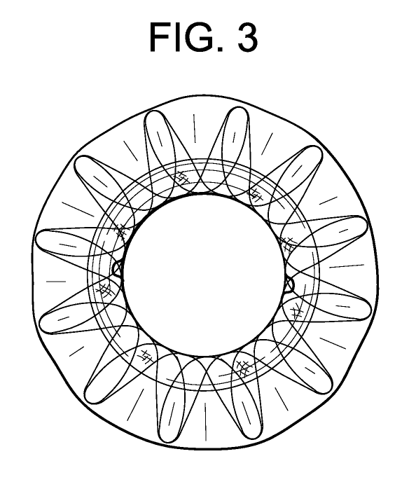

FIG. 3 is a perspective view of one embodiment showing the multi-component cuff or atrial sealing gasket wherein the inner section is synthetic material such as PET or similar fabric, and the outer section is composed of stabilized tissue. FIG. 3 shows the valve without the leaflets installed. FIG. 1 shows how the collar is composed of at least two sections, and inner and an outer section. Here, the inner section is made from synthetic material, such as PET or Dacron®. The outer section is made from stabilized tissue. The inner fabric/synthetic material section is important in preferred embodiments in order to facilitate ingrowth once the valve is deployed within the patient.

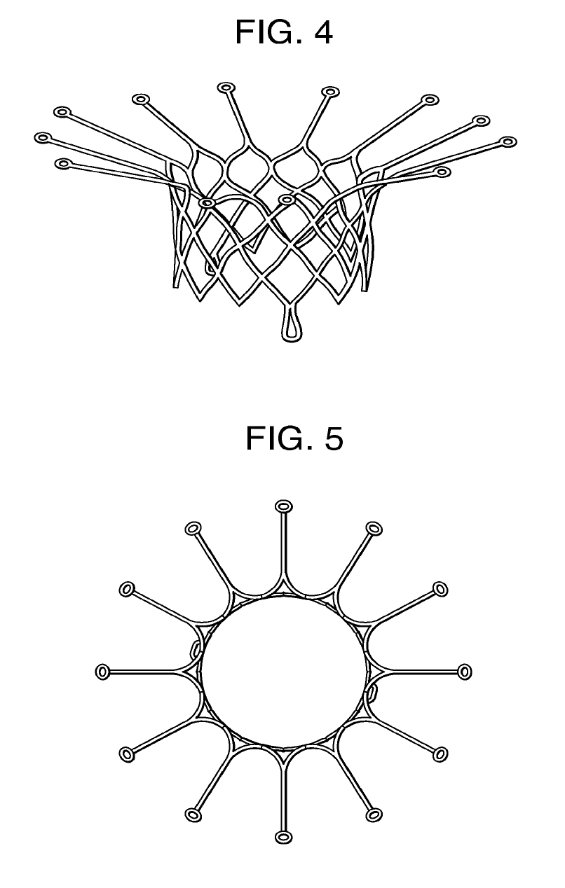

FIG. 4 is a side view of one embodiment showing the articulating collar support structures of the flared end of the tubular stent. Note this figure does not illustrate the final valve product as it has neither the surface coatings, e.g. synthetic material and/or stabilized tissue, nor internal leaflet structures have been added.

FIG. 5 is a top view of one embodiment showing the articulating collar support structures of the flared end of the tubular stent. Note this figure does not illustrate the final valve product as it has neither the surface coatings, e.g. synthetic material and/or stabilized tissue, nor internal leaflet structures have been added.

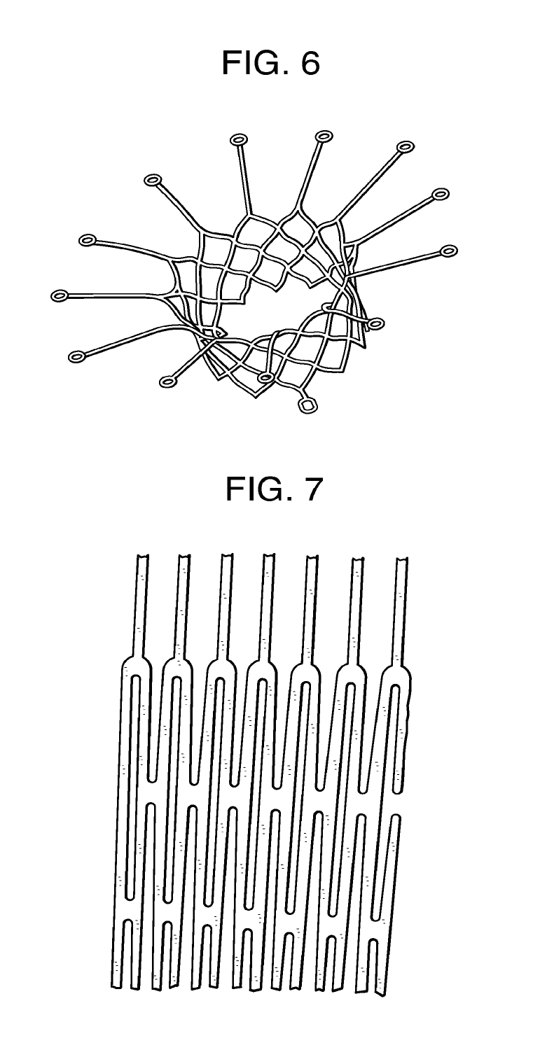

FIG. 6 is a perspective view of one embodiment showing the articulating collar support structures of the flared end of the tubular stent. Note this figure does not illustrate the final valve product as it has neither the surface coatings, e.g. synthetic material and/or stabilized tissue, nor internal leaflet structures have been added.

FIG. 7 is an illustration of a close-up of the stent construction at the junction where the stent body transitions to the articulating radial structures. FIG. 4 shows detail of a compacted, or un-expanded (not deployed) stent structure.

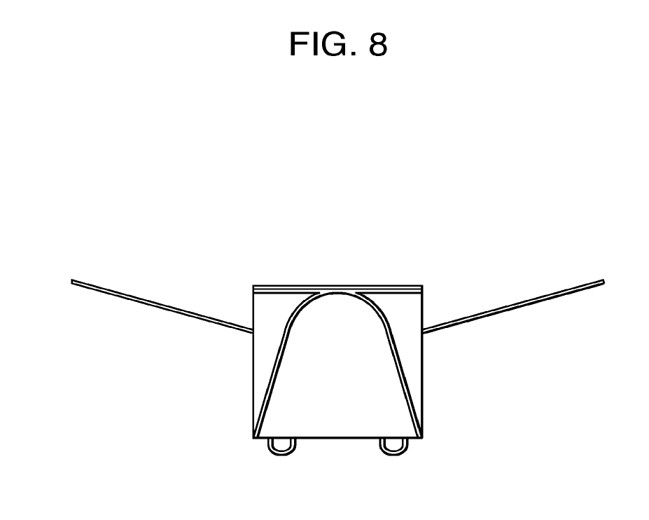

FIG. 8 is an illustration of a side view with false-transparent detail of the inner leaflet structure location of an embodiment of the present invention showing how the articulating feature may be used to create a valve having the leaflet structures high in the stent body and raised into the atrial space while maintaining a collar support and sealing originating lower on the stent body.

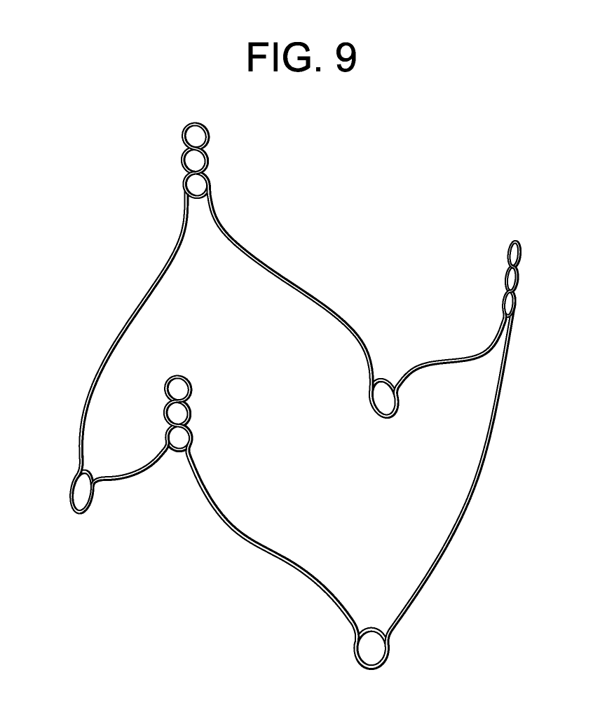

FIG. 9 is an illustration of a perspective view of an expandable wire frame for a prosthetic valve. The frame consists of three arches, wherein the base of each arch is a single spring-form circular connector, and wherein each arch is connected at each apex by a spring-form connector comprising three stacked circles of equal diameter. Each side of each arch is comprised of wire formed into a shallow “S” shape.

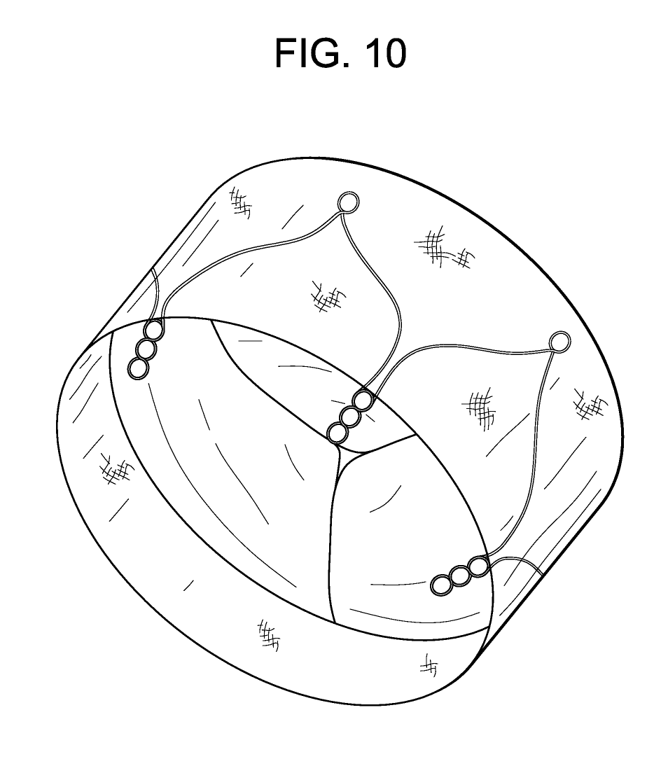

FIG. 10 is an illustration of a leaflet assembly comprising the wire frame of FIG. 9 overlaid by stabilized tissue or biocompatible fabric, such leaflet assembly centered within a self-expanding, circular metallic stent.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention.

Accordingly, the scope of the invention is determined by the scope of the following claims and their equitable Equivalents.

What is claimed is:

1. A body for a prosthetic cardiovascular valve, comprising:
a body portion having a proximal end and a distal end; and
a collar support portion integrally formed with and extending radially outwardly from the distal end of the body portion, the collar support portion including a plurality of radially extending tines.

2. The body of claim 1, wherein the body portion and the collar support portion are formed of a superelastic metal alloy.

3. The body of claim 1, wherein at least one of the plurality of radially extending tines terminates in a loop.

4. The body of claim 1, wherein the body portion is substantially cylindrical.

5. The body of claim 1, wherein the body portion is D-shaped in cross-section.

6. The body of claim 1, wherein the plurality of radially extending tines includes at least six tines.

7. The body of claim 1, wherein the plurality of radially extending tines includes at least ten tines.
